(19) United States
(12) Patent Application Publication
(10) Pub. No.: US 2006/0241069 A1
(43) Pub. Date: Oct. 26, 2006

(54) Treatment of hyperproliferative disease

(75) Inventors: Pnina Fishman, Herzliya (IL); Lea Madi, Rishon Le Zion (IL); Sara Bar Yehuda, Rishon Le Zion (IL)

Correspondence Address: BROWDY AND NEIMARK, P.L.L.C.;624 NINTH STREET, NW, SUITE 300, WASHINGTON, DC, 20001-5303, US

(73) Assignee: Can-Fite Biopharma Ltd., Petach Tikva (IL)

(21) Appl. No.: 10/529,650

(22) PCT Filed: Feb. 24, 2005
(86) PCT No.: PCT/IL05/00232

§ 371 Date: Mar. 31, 2005

Related U.S. Application Data
(60) Provisional application No. 60/547,561, filed on Feb. 26, 2004.

(51) Int. Cl. A61K 48/00; C07H 21/02

(52) U.S. Cl. 514/44; 536/23.1

(57) ABSTRACT

A short interfering RNA (siRNA) duplex that includes complementary sense and anti-sense sequences corresponding to at least part of the A3 adenosine receptor (A3AR) mRNA sequence, a double-stranded RNA (dsRNA) construct that can be converted within a cell into a siRNA duplex and a transcript system that can induce transcription within cells of either a siRNA duplex or a dsRNA construct Also disclosed are a method and a pharmaceutical composition for treating a hyperproliferative disease.

### FIELD OF THE INVENTION

This invention is generally in the field of RNA interference (RNAi) and concerns small interference RNA (siRNA) duplexes, RNA constructs that serve as precursors for said duplexes, expression systems that can direct the synthesis of the siRNA duplexes or said constructs within cells as well as the use of the siRNA duplexes, constructs or expression systems in the treatment of hyperproliferative diseases.

### BACKGROUND OF THE INVENTION

The following is a list of prior art which is considered to be pertinent for describing the state of the art in the field of the invention. Acknowledgement of these references herein will be made by indicating the number from their list below within brackets.
1. Gessi, S., Varani, K., Merighi, S., Morelli, A., Ferrari, D., Leung, E., Baraldi, P. G., Spalluto, G., and Borea, P. A. A(3) adenosine receptors in human neutrophils and promyelocytic HL 60 cells: a pharmacological and biochemical study. Br. J. Pharmacol., 134:116-126, 2001.
2. Merighi, S., Varani, K., Gessi, S., Cattabriga, E., lannotta, V., Ulouglu, C., Leung, E., and Borea, P. A. Pharmacological and biochemical characterization of adenosine receptors in the human malignant melanoma A375 cell line. Br. J. Pharmacol, 134:1215-1226, 2001.
3. Suh, B. C., Kim, T. D., Lee, J. U., Seong, J. K., and Kim, K. T. Pharmacological characterization of adenosine receptors in PGT-beta mouse pineal gland tumour cells. Br. J. Phannacol., 134:132-142, 2001.
4. Dixon, A. K., Gubitz, A. K., Sirinathsinghji, D. J., Richardson, P. J., Freeman, T. C., Tissue distribution of adenosine receptor mRNAs in the rat. Br. J. Phannacol., 118:1461-1468, 1996.
5. Fishman, P., Bar-Yehuda, S., Ohana, G., Barer P., Ochaion A., Erlanger and Madi, L., An agonist to the A₃ adenosine receptor inhibits colon carcinoma growth in mice via modulation of GSK-3βand NF-κB. Oncogene, 23: 2465-2471, 2003.
6. Andino R. RNAi puts a lid on virus replication. Nat. Biotech. 21: 629-30, 2003.
7. Carmichael G. G. Silencing viruses with RNA. Nature 418: 379-80, 2002.
8. Gitlin, L., Karelsky, S., Andino, R. Short interfering RNA confers intracellular antiviral immunity in human cells. Nature 418: 430-33, 2002.
9. Jacque J. M., Triques, K., Stevenson M. Modulation of HIV-1 replication by RNA interference. Nature 418: 435-38, 2002.
10. US patent application 2004/0001811.
11. U.S. patent application Ser. No. 60/420,038.
12. Madi, L., S. Bar-Yehuda, F. Barer, E. Ardon, A. Ochaion, and, P. Fishman. 2003. A3 adenosine receptor activation in melanoma cells: association between receptor fate and tumor growth inhibition. J. Biol. Chem. 278:42121-42130.

Adenosine receptors are classified into four major groups: A1, A2a, A2b and A3. It has been shown that the A3 adenosine receptor (A3AR) is abundantly expressed in tumor vs. normal cells. High receptor expression was found in different tumor cell lines, including Jurkat T, pineal gland, breast cancer, prostate cancer and melanoma, whereas in normal cells low expression was reported (1-4). Activation of this receptor with the highly selective A3 agonist IB-MECA, resulted in growth inhibition of various neoplastic cells including melanoma, colon and prostate carcinoma. Recent studies have demonstrated that PKAc phosphorylates and inactivates GSK-3β. IB-MECA alters the expression of GSK-3βand β-catenin, key components of the Wnt signaling pathway. Consequently it led to inhibition in the expression of the cell cycle progression genes c-myc and cyclin D1 (5).

Introduction of double-stranded oligoribonucleotides results in degradation of the target endogenous MRNA by a mechanism which is highly sequence specific. This mechanism of RNA interference (RNAi) employs short pieces of dsRNA, called small interfering RNA (siRNA) which are approximately 21-23 nucleotides long and which include a sequence which is complementary to the sequence of the target MRNA. Attempts have been made to inhibit viral replication in human cell cultures, such as HIV, polio and hepatitis B & C, using siRNA (6-9). It was also proposed to employ this approach in inhibiting expression of apoptotic genes (10).

### SUMMARY OF THE INVENTION

It is an object of the present invention to provide a polynucleotide based therapy to control hyperproliferative diseases.

In accordance with the present invention it was found that by employing the RNA interference (RNAi) principle to block the expression of the A3 adenosine receptor (A3AR), proliferation of proliferating or hyperproliferating cells can be inhibited. The proliferating cells may be cells that over-express the A3AR, such as inflammatory cells and cancer cells (see U.S. patent application Ser. No. 60/420,038). In accordance with one embodiment of the invention, to be referred to herein at times as the “siRNA duplex embodiment”, an siRNA duplex that includes complementary sense and anti-sense sequences corresponding to at least part of the A3AR messenger RNA (mRNA) or an alternative splice form, mutant or cognate thereof is provided and utilized. In accordance with another embodiment, to be referred to herein at times as the “precursor embodiment” a dsRNA construct that can be converted within the cell, e.g. by the RNAse III Dicer, into said siRNA duplex (hereinafter “siRNA precursor”) is provide and utilized. In accordance with a further embodiment, to be referred to herein at times as the “transcription system embodiment” a transcript system that can induce the transcription within cells of said siRNA duplexes or siRNA precursor is provided and utilized.

The term “dsRNA” as used herein denotes an RNA construct that has a major portion that is double-stranded. A dsRNA may be constructed out of two independent oligonucleotides that are hybridized to one another. Alternatively it may be comprised of a single oligonucleotide having complementary sequences that folds in a manner such that said complementary sequences hybridize to one another, typically to form a hairpin-type construct. Such dsRNA may have terminal regions or hairpin loop regions in which the nucleotide sequence are not paired and are thus single-stranded. Also, occasionally the double-stranded portion of the dsRNA may contain one or more mismatched nucleotides that are not paired (hybridized) with a complementary one.

The term “A3AR mRNA” as used herein denotes an mRNA that encodes for the A3AR.

The term “siRNA duplex” denotes an ensemble of one or two oligonucleotides including a sense and an antisense sequences that can hybridize to form a dsRNA. In the siRNA duplex in accordance with the invention the sense and the antisense sequences correspond to at least part of the A3AR MRNA sequence. The siRNA duplex, in accordance with one embodiment, includes two independent oligonucleotides, one with a sense sequence and the other with an antisense sequence; in accordance with another embodiment it includes one oligonucleotide with both the sense and the antisense sequences, typically separated from one another by several nucleotides such that said one oligonucleotide can fold to hybridize with one another. Preferably, the sense or antisense sequences sufficiently correspond to at least part of the A3AR MRNA sequence so as to activate RNA interference-based cleavage of the MRNA sequence.

The term “corresponding” when used to refer to the relationship between the siRNA duplex and the A3AR mRNA means that the sense sequence and the antisense sequence have a high degree of identity or complementarity, respectively, to a selected sequence in the A3AR niRNA. In some cases this means that one or both of the sense or the antisense sequence may be completely identical or complementary, respectively, to the selected sequence. In other cases, while most (typically more than 90%) of the nucleotides in the two sequence with have a counterpart in said selected sequence, one or both of the sense and the antisense sequences may include one or more nucleotides that have no counterpart in the selected sequence.

The term “active agent” as used herein denotes the siRNA duplex, in the case of the siRNA duplex embodiment; the siRNA precursor in the case of the precursor embodiment; and the transcription system in the case of the transcription system embodiment. The active agent in accordance with the invention may be used for treating a hyperproliferating disease including cancer, a benign hyperplastic condition, an inflammatory disease and others.

The term “target cells” as used herein denotes the undesired hyperproliferating cells, the hyperproliferation of which is associated with a disease to be treated and which are thus the target for the proliferation inhibition therapy in accordance with the invention. According to specific embodiments of the invention the target cells are tumor cells, including solid tumors such as carcinoma, sarcoma or melanoma as well as blood tumors including lymphoma or leukemia; or inflammatory cells such as neutrophils or mast cells. The disease to be treated being accordingly cancer and inflammatory disease, respectively.

The present invention may be applied in the treatment of diseases that are associated with target cells that over-express the A3AR. Examples of such diseases are cancer and inflammatory diseases. Some benign hyperplastic conditions, such as adenoma, benign prostate hyperplasia and others are also associated with over-expression of A3AR and accordingly lend themselves for treatment in accordance with the invention.

The siRNA duplexes, either introduced into the target cells directly in accordance with the siRNA duplex embodiment or formed within the target cells by either the precursor embodiment or the expression system embodiment, silence the expression of the A3AR in the target cells. As can be appreciated, the silencing of the A3AR expression according to the siRNA duplex embodiment and the precursor embodiment of the invention is usually transient and the target cells in such a case need to be repeatedly contacted with the respective active agent. However, the silencing of the A3AR expression according to the transcription system embodiment is typically longer lasting and at times also permanent and accordingly the target cells need to be contacted with the respective active agent with a lesser frequency, at times only once in the course of a treatment. The artisan should be able to determine in appropriate animal or human trials, the proper treatment regimen in each case.

For therapeutic use, the active agent in accordance with the siRNA duplex embodiment or the precursor embodiment may be contacted with the target cells either in vivo or ex vivo under conditions which will permit the active agent to enter and transfect the target cells and silence the expression of the A3AR in them. Any compositions and procedures applicable for ex vivo transfection of such active agents, or any delivery systems, e.g. liposome delivery systems applicable for systemic delivery of such active agents to the target cells for in vivo transfection are known per se.

The A3AR-specific siRNA duplex includes a sense sequence and antisense sequence that can hybridize to one another, as aforesaid, to form a double stranded RNA stretch, which corresponds to at least a part of the A3AR mRNA. The siRNA precursor may be a long precursor RNA that is cleavable in the cell, e.g. by RNAse Dicer processes, to siRNAs, at least one of them being an A3AR-specific siRNA duplex with sense and antisense sequences that can form said double-stranded RNA stretch. The reference in the following to a “double-stranded stretch” relates to the complementary sense and antisense sequences of the siRNA duplex that, once hybridized, form said double-stranded RNA stretch. Reference is made to a double-stranded stretch for ease of description as it is clear that depending on the conditions the sense and the antisense sequences of the siRNA duplex may either hybridize to form said double-stranded stretch or remain non-hybridized. The double-stranded stretch should preferably be less than 25 nucleotides in length. In the double-stranded stretch, at least one of the sense or the antisense sequences may comprise a nucleotide overhang of 1 to 4 nucleotides, preferably 2 or 3 nucleotides in length. In one embodiment, the nucleotide overhang is on the 3′-terminus of the antisense RNA strand, and the 5′-end is blunt. The antisense RNA strand and sense RNA strand have a corresponding region, which may be 19 to 24 nucleotides, preferably 21 to 24 nucleotides, and most preferably 22 nucleotides in length. The antisense RNA strand may be less than 30, preferably less than 25, and most preferably 21 to 24 nucleotides in length. In one embodiment, the sense and the antisense sequences are chemically linked to one another. The chemical linker may be an oligonucleotide linker with 3 to 15, preferably less than 10 and more than 4 nucleotides, hexamethylene glycol linker, apoly-(oxyphosphinico-oxy-1,3-propanediol) linker, or an oligoethyleneglycol linker.

The nucleotides of the double-stranded region of the siRNA duplex or of the siRNA precursor according to the duplex embodiment or the precursor embodiment, respectively, may be fully paired. Alternatively, the double-stranded region of the dsRNA may also contain one or more, e.g. 1 to 7 mismatches or bulges. In case of a mismatch, typically one of the nucleotides may be guanine and the other uracil.

A siRNA duplex according to an embodiment of the invention have at least one, preferably more than one, and most preferably most of the following features:
a) the sense and antisense sequences in the siRNA duplex that can pair with one another includes approximately 20 to 22 nucleotides within the coding region of the A3AR mRNA;
b) at least one, and preferably both, of said sense or antisense sequences in the siRNA duplex have a short tail of poly T;
c) at least one, and preferably both, of said sense or antisense sequences in the siRNA duplex have no G-nucleotide at the 3′end to prevent digestion by RNases;
d) the GC contents is less than 50%; and
e) the sequences are be unique to the A3 adenosine receptor and have no similarity to the other subtypes of adenosine receptors (A1, A2A and A2B ).

The transcription system, according to the transcription system embodiment, may include one or two plasmids or viral vectors, which include a sequence, typically under control of a suitable promoter that is transcribed into an RNA oligonucleotide of the siRNA duplex or siRNA precursor of the invention within cells transected by it. The target cells may be contacted with said expression vector in vivo or in vitro. The promoter may be a constitutively expressed promoter or a promoter that is preferentially inducible under conditions prevailing in the target cells. The promoter may, for example, be a pol II, pol III or an H1 promoter.

By one embodiment, said transcription system comprises an antisense coding DNA that can be transcribed into an antisense RNA sequence corresponding to a selected region of the A3AR mRNA, a sense coding DNA that can be transcribed into a sense RNA sequence of the same region of the A3AR mRNA, and one or more promoters capable of inducing transcription of said antisense and sense RNAs from said antisense and sense coding DNAs, respectively. The transcribed RNA oligonucleotides may constitute an siRNA duplex or an siRNA precursor. The siRNA duplex or siRNA precursor may be transcribed as one continuous sequence to subsequently form a hairpin type dsRNA. Alternatively, the sense and the antisense sequences may be independent transcribed sequences that can hybridize to yield a dsRNA. As will be appreciated, the sense sequence and the antisense sequence may be located both on the same DNA vector or, alternatively, each in a separate DNA vector.

There are numerous ways to construct viral or plasmid vectors to form transcription systems according to the invention and to utilize them to transfect target cells. Any suitable such method may be applicable in accordance with the invention.

The invention also provides a method for treating a hyperproliferative disease manifested by an undesirable increase in proliferation of cells, comprising contacting at least a portion of said cells with an active agent which interferes with the expression of the A3AR, the active agent being selected from the siRNA duplex, the dsRNA construct or the transcript system of the invention.

According to another aspect of the invention, there is provided a pharmaceutical composition for treating a hyperproliferative disease manifested in undesired increased proliferation of cells, comprising an effective amount of an active agent which interferes with the expression of the A3AR, the active agent being selected from the siRNA duplex, the dsRNA construct or the transcript system of the invention and a pharmaceutically acceptable carrier. The pharmaceutical composition optionally includes one or more agents that can facilitate incorporation of said polynucleotide molecule into the hyperproliferating target cells.

According to a further aspect, the present invention provides use of an active agent which interferes with the expression of A3AR, the active agent being selected from the siRNA duplex, the dsRNA construct or the transcript system of the invention, for the manufacture of a pharmaceutical composition for treating a hyperproliferative disease manifested in undesired increased proliferation of cells.

According to a still further aspect, the present invention provides a method for inhibiting expression of the A3AR gene in target cells comprising introducing an active agent into said target cells, wherein said active agent is selected from the siRNA duplex, the dsRNA construct or the transcript system of the invention. In a preferred embodiment, the A3AR gene expression is inhibited by at least 25%. In another preferred embodiment, the active agent is transfected into the target cells by a delivery system. Also provided is the use of an active agent, selected from the siRNA duplex, the dsRNA construct or the transcript system of the invention in the preparation of a pharmaceutical composition for use in a method for inhibiting expression of the A3AR gene in target cells.

According to a still further aspect, the present invention provides a method of activating A3AR-specific RNA interference in a target cell comprising introducing into said target cells an active agent, wherein said active agent is selected from the siRNA duplex, the dsRNA construct or the transcript system of the invention. Also provided is the use of an active agent, selected from the siRNA duplex, the dsRNA construct or the transcript system of the invention in the preparation of a pharmaceutical composition for use in a method of activating A3AR-specific RNA interference in a target cell.

The term “effective amount” in the context of the present invention refers to an amount of said active agent, which is effective in reducing proliferation of hyperproliferative cells such as tumor cells in cancer and inflammatory cells in inflammatory diseases. The “effective amount” can be readily determined, in accordance with the invention, by administering to a plurality of tested subjects various amounts of the polynucleotide and then plotting the physiological response as a function of the amount. Alternatively, the effective amount may also be determined, at times, through experiments performed in appropriate animal models and then extrapolating to human beings using one of a plurality of conversion methods. As known, the effective amount may depend on a variety of factors such as mode of administration; the age, weight, body surface area, gender, health condition and genetic factors of the subject; other administered drugs; etc.

The pharmaceutical composition, and particularly that according to the siRNA duplex and the precursor embodiments, may typically comprise less than 5 mg of the active agent, preferably in a range of 0.0001 to 2.5 mg, more preferably 0.1 to 200 μeg, even more preferably 0.1 to 100 μg, and most preferably less than 25 μg per kilogram body weight of the subject. The pharmaceutically acceptable carrier may be an aqueous solution, such phosphate buffered saline. The pharmaceutically acceptable carrier may comprise a micellar structure, such as a liposome, capsid, capsoid, polymeric nanocapsule, or polymeric microcapsule. In a preferred embodiment, the micellar structure is a liposome. The pharmaceutical composition may be formulated to be administered by inhalation, infusion, orally or by injection, preferably by intravenous or intraperitoneal injection.

Also provide are a recombinant plasmid comprising nucleic acid sequences for expressing one or more of the sequences comprising the siRNA duplex of the invention, and a kit comprising reagents for activating A3AR-specific RNA interference in a cell or organism.

The details of some embodiments of the invention are set forth in the accompanying drawings and the description below. Other features, objects, and advantages of the invention will be apparent from the description and drawings, and from the claims.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows an RT-PCR analysis of A₃AR expression in mouse B16-F10 melanoma cells transfected with the two different siRNA duplexes (siRNA1, which is an siRNA sequence directed to the mouse A₃AR and siRNA2, which is directed to the human A₃AR), 30 min., 1 h and 2 h after transfection, utilizing TM-buffer treated cells as a control and β-actin as an internal control

FIG. 2 shows a western blot (WB) analysis of A₃AR expression in the mouse melanoma cells transfected with siRNA1 or siRNA2 duplexes, 24 h and 48 h after transfection.

FIG. 3 demonstrates the exhibition of A₃AR (highlight) in the B16-F10 melanoma cells transfected with siRNA1 utilizing confocal microscopy.

FIG. 4 shows the proliferation of B16-F10 melanoma cells transfected with siRNA1 and siRNA2 duplexes, tested by [³H] thymidine incorporation assay after 24 h. Results are presented as mean±SE.

FIG. 5 shows metastatic melanoma foci developed in lung of mice, fifteen days after intravenous (iv) inoculation of siRNA treated B16-F1 cells (2.5×10⁵).

FIG. 6 shows the number of lung metastatic melanoma foci, with results being presented as mean±SE.

FIG. 7 shows the A₃AR mRNA expression (determined by RT-PCR) in human HCT-116 colon carcinoma cells and LnCap prostate carcinoma cells transfected with siRNA2 and siRNA3 duplexes homologous to the human A₃AR, 1 h after transfection;

FIG. 8 is a WB analysis of A₃AR expression level in HCT-116 cells and LNCaP prostate carcinoma cells transfected with siRNA2 and siRNA3 duplexes, 24 h after transfection. β-actin was used as an internal control;

FIG. 9 shows the proliferation of HCT-116 and LNCaP cells transfected with siRNA2 or siRNA3 duplexes, examined by [³H] thymidine incorporation assay 24 h after transfection. Results are presented as mean±SE;

FIG. 10 shows the cAMP level in HCT-116 and LNCaP cells transfected with siRNA2 and siRNA3. Results are presented as mean±SE.

FIG. 11 shows an RT-PCR analysis of ICER in HCT-116 and LNCaP cells transfected with siRNA2 and siRNA3;

FIG. 12 shows an RT-PCR analysis of cyclin D1 expression in HCT-116 and LNCaP cells 1 h after transfection with siRNA2 and siRNA3.

FIG. 13 shows the Cyclin D1 protein expression level in HCT-116 and LNCaP cells 24 h after transfection with siRNA2 and siRNA3.

FIG. 14 shows an example of a designed oligonucleotide to be included eventually in a plasmid in accordance with the transcription system embodiment; and

FIG. 15 shows the design of a plasmid containing an A3AR-specific siRNA sequence in accordance with the transfection system embodiment of the invention.

### DETAILED DESCRIPTION OF SPECIFIC EMBODIMENTS

Materials and Methods:

siRNA Transfection

The sequence for targeting the mouse (Mus musculus) adenosine A₃ receptor (accession No-AF069778), siRNA1, corresponded to the coding region 360-380 (5′-AACGGTTACCACTCAAAGAAG [SEQ ID No:1]) relative to the start codon. The siRNA1 duplex containing the following sequence with the sense and antisense was used:

| | |
|---|---|
| r(CGGUUACCACUCAAAGAAG)d(TT), | [SEQ ID No: 2] |
| and | |
| r(CUUCUUUGAGUGGUAACCG)d(TT). | [SEQ ID No: 3] |

Two sequences for targeting human adenosine A₃ receptor were designed. The first sequence, siRNA2, corresponded to the coding region 1843-1863 (5′-AAGTGACCCACCTGTGATGAG [SEQ ID No:4 ]) relative to the start codon. The siRNA2 duplex with the following sense and antisense sequences was used:

| | |
|---|---|
| r(GUGACCCACCUGUGAUGAG)d(TT), | [SEQ ID No: 5] |
| and | |
| r(CUCAUCACAGGUGGGUCAC)d(TT). | [SEQ ID No: 6] |

The second sequence for targeting human adenosine A₃ receptor, siRNA3, corresponded to the coding region 1077-1097 (5′-AAGGGTGCCTAGTTGACTTAC [SEQ ID No:7] relative to the start codon. The siRNA3 duplex with the following sense and antisense sequences were used:

| | |
|---|---|
| r(GGGUGCCUAGUUGACUUAC)d(TT) | [SEQ ID No: 8] |
| and | |
| r(GUAAGUCAACUAGGCACCC)d(TT). | [SEQ ID No: 9] |

siRNA transfection was performed by utilizing TransMessenger (TM) transfection reagent according to the manufacturer's instructions (Qiagen). Briefly, 1×10⁵ tumor cells (B16-F10 mouse melanoma; HCT-116 human colon carcinoma; LN-CaP human prostate carcinoma) were seeded in 24-well plate supplemented with 1 ml RPMI and 10% FBS (Beit Haemek, Haifa, Israel) for 24 h at 37° C. in 5% CO₂. Then cells were serum starved for 24 h. Following the starvation the growth medium was aspirated gently and the adherent cells were carefully washed with PBS. Washed cells were then supplemented with 0.3 ml growth medium without serum and 106 μl of the transfection complex (3.2 μl enhancer, 91.4 μl EC-R buffer and 1.6 μg siRNA) was added. Cells were incubated with the transfection complex for 3 h at 37° C. and 5% CO₂. The transfection complexes were washed with PBS and the cells were supplemented with RPMI containing 10% serum for different time periods. At the end of the incubation period protein and total RNA were extracted for Western blot (WB) and RT-PCR analysis.

Plasmid Transfection

0.8 μg of mammalian expression vector pcDNA3.1 carrying A₃AR (Guthrie cDNA Resource Center) was transfected to 2×105 adherent HEK-293 or HCT-116 cells utilizing Lipofectamine™ 2000 reagent according to manufacture (Invitrogen) instructions. Stably transfected HCT-116 cells were grown in RPMI with 10% FBS and 0.6 mg/L G418. Stably transfected HEK-293 were grown in DMEM with 10% FBS and 0.6 mg/L G418 for 4-weeks, than proteins or RNA were extracted for western and RT-PCR analysis.

Cell Proliferation Assay

Following transfection, 10,000 cells per well were seeded in 96-well plates in RPMI with 1% serum for 24 hours. For the last 18 h of incubation, each well was pulsed with 1 μCi [³H]-thymidine. Cells were harvested and the [³H]-thymidine uptake was determined in a liquid scintillation counter (LKB).

Protein and RNA Analysis

Protein extracts from whole cells, nucleus or cytosol, were subjected to WB analysis. Protein concentrations were determined using the Bio-Rad protein assay dye reagent. Equal amounts of the sample (50μg) were separated by SDS-PAGE, using 12% polyacrylamide gels. The resolved proteins were then electroblotted onto nitrocellulose membranes (Schleicher & Schuell, Keene, N.H., USA). Membranes were blocked with 1% bovine serum albumin and incubated with the desired primary antibody (dilution 1:1000) for 24 h at 4° C. Antibodies utilized for WB analysis: murine (R-18) and human A₃AR (C-17), actin (I-19) were purchased from Santa Cruz Biotechnology Inc., CA. Antibodies to murine and human cyclin D1 were from Upstate Biotechnology, Lake Placid, N.Y. phospho-CREB (serine 133) were from Chemicon, Temcula, Calif.

Bands were recorded using BCIP/NBT color development kit (Promega). The optical density of the bands was quantified using an image analysis system and corrected by the optical density of the corresponding actin bands.

Total RNA (1 μg) extracted with Tri-reagent (Sigma) was used for the RT reaction which was performed at 47° C. for 30 min. The RT-PCR was performed by utilizing the SuperScript™ One-Step RT-PCR with Platinum Taq (Invitrogen) according to the manufacturer's instructions. The condition for RT-PCR was depended on the primers used. For amplification of human A₃AR the primers 5′-ACGGTGAGGTACCACAGCTTGTG [SEQ ID No:10] and 3′-ATACCGCGGGATGGCAGACC [SEQ ID No:11] were used. The RT was followed by PCR reaction i.e., heating to 94° C. for 2 min., 30 cycles of 94° C. for 15 s, 61 C. for 30 s and 68° C. for 2 min. For amplification of mouse A₃AR the primers 5′-CCGAGAAGGGGAAGACAGG [SEQ ID No:12] and 3′- TGCTATATTCTTCCCCCAAG [SEQ ID No:13] were used. The PCR conditions were as follows: heating to 94° C. for 2 min, 25 cycles of 94° C. for 15 s, 56° C. for 30 s and 68° C. for 2 min. For amplification of human cyclin D1 the primers 5′-GAACAAACAGATCATCCGCAAACAC [SEQ ID No:14] and 3′-GCTCCTGGCAGGCCCGGAGGCAGT [SEQ ID No:15] were used. The PCR conditions were as follows: heating to 94 C. for 2 min, 25 cycles of 94° C. for 15 s, 58° C. for 30 s and 68° C. for 2 min. For amplification of the mouse cyclin D1 the 5′-AACTTCCTCTCCTGCTACCG [SEQ ID No: 16] and 3′-GTGGCTCCCGCCTGCCCGGT [SEQ ID No: 17] were used.

Immunostaining and Confocal Microscopy

B16-F10 murine melanoma cells were grown for 24 h on cover slips (coated with poly-L-lysine, 500 μg/ml) in RPMI with 10% FBS for overnight. Cells were serum starved for 24 h, washed and subjected to siRNA transfection for 3 h as described above. siRNA treated and non treated cells were then washed, immunostained with A₃AR, and murine Cy3-conjugated anti-goat IgG (Chemicon, Temcula, Calif.) and visualized by confocal microscopy as described

cAMP Assay

cAMP level in siRNA treated and the control cells was assayed as described (12) by utilizing a commercial enzyme linked immunosorbent assay kit based on competitive binding assay (R&D systems).

In Vivo Studies

Male C57BL/6J mice (Harlan Laboratories) aged 2 months, weighing an average of 20 g were used. Standardized pelleted diet and tap water were supplied. All the experiments were performed in accordance with the UKCCCR guidelines (Workman, P., A. et al. 1998. United Kingdom Co-ordinating Committee on Cancer Research (UKCCCR) guidelines for the welfare of animals in experimental neoplasia (Second Edition). Br. J. Cancer 77:1-10) and Can-Fite Animal Care and Use Committee, Petach Tikva, Israel. In the artificial lung metastatis model, 2.5×10⁵ siRNA treated or control B16-F10 melanoma cells were inoculated to mice intravenously. Each group contained 10 mice.

Mice were sacrificed after fifteen days, lungs removed and the black metastatic foci were counted using a Dissecting Microscope.

Statistical Analysis

siRNA transfection experiments of each cell type were carried out at least 3 times. Proliferation and cAMP data are a summary of at least 3 different experiments. WB and RT-PCR data are representative of one out of 3 experiments. The statistical analysis for the in vivo study was carried out among the different individuals in each group. Statistical analysis was carried out utilizing the Student's t-test.

Results

siRNA Silencing Suppresses In Vitro and In Vivo Melanoma Growth

Three different siRNA duplexes to interfere with receptor expression were designed, siRNA1 to target A₃AR in the mouse B16-F10 melanoma cells and siRNA2 or siRNA3 to target A₃AR in the human HCT-116 colon and LNCaP prostate carcinoma cells.

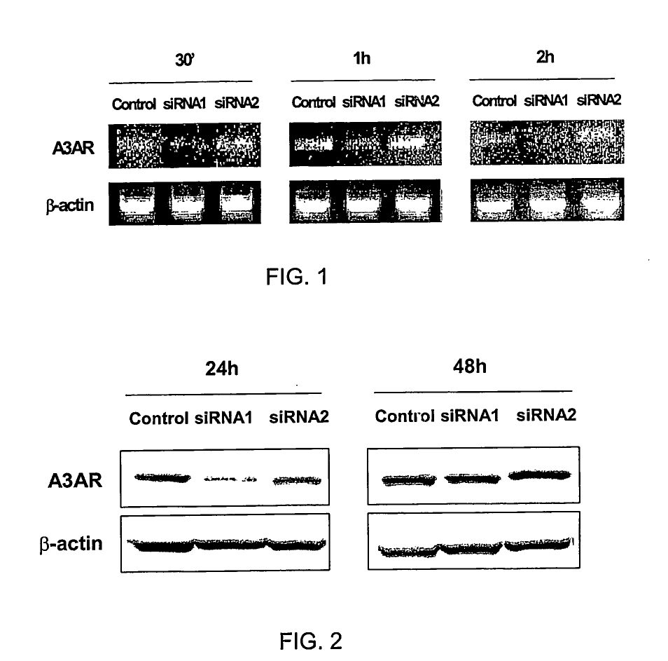

B16-F10 melanoma cells were transfected with siRNA1 and siRNA2 (the former corresponding to the mouse A₃AR and the latter, serving as a control, to the human A₃AR). Results show that the down-regulation in MRNA level of the cells treated with siRNA1 but not with siRNA2 reduced the level of the endogenous A₃AR mRNA (FIG. 1). In accordance with these data it can be seen that only the siRNA1 duplex reduced A₃AR protein expression level whereas the siRNA2 duplex did not change receptor level. The suppression lasted for 24 h and after 48 h, A₃AR protein level was fully restored to that of the control cells (FIG. 2).

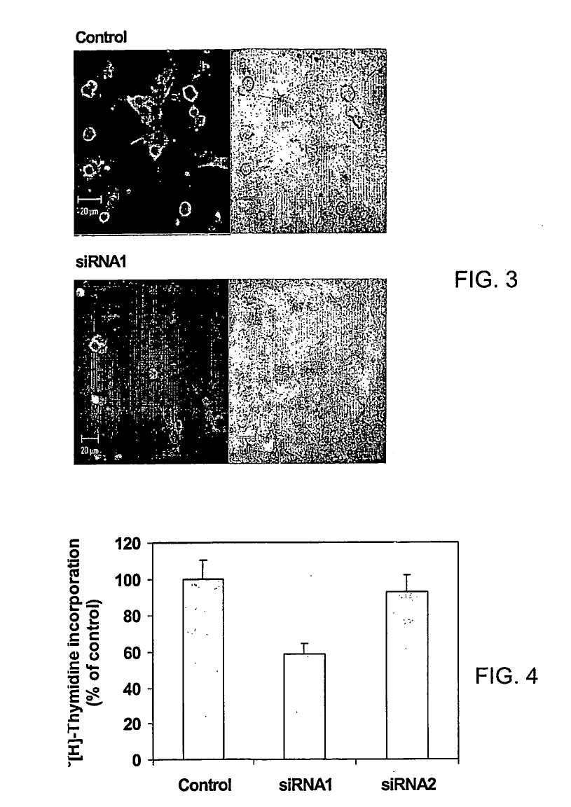

These results were further confirmed by confocal laser microscopy examination in which less expression of A₃AR in cells transfected with siRNA1 compared to the control cells was observed (FIG. 3).

The effect of siRNA-induced A₃AR knock-down on melanoma cell growth was tested by monitoring the proliferation of the cells utilizing [³H] thymidine incorporation assay. Treatment with siRNA1 had statistically significant inhibitory effect (40.3%±6.5%) on cell proliferation at 24 h post transfection compared to control siRNA (siRNA2) treated cells (FIG. 4).

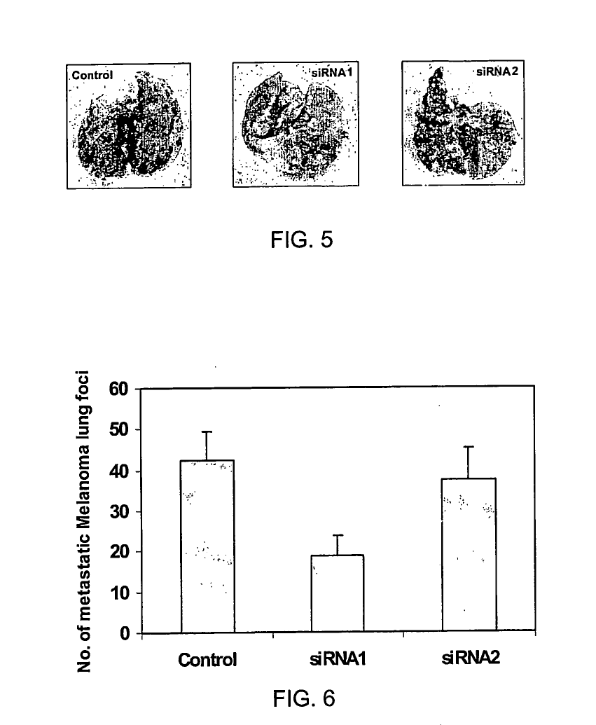

The effect of A₃AR siRNA on regulating cell proliferation was further tested in vivo by inoculating C57B1/6J mice with melanoma cells pre-treated with siRNA1, siRNA2 and cells treated with vehicle only (“control”). In the group inoculated with siRNA1 transfected cells, marked decrease in the number of melanoma foci was seen (56%±5.3% inhibition, p<0.001 in comparison to the control group) (FIGS. 5 and 6).

Inhibition of Colon and Prostate Carcinoma Growth Upon A3AR Silencing

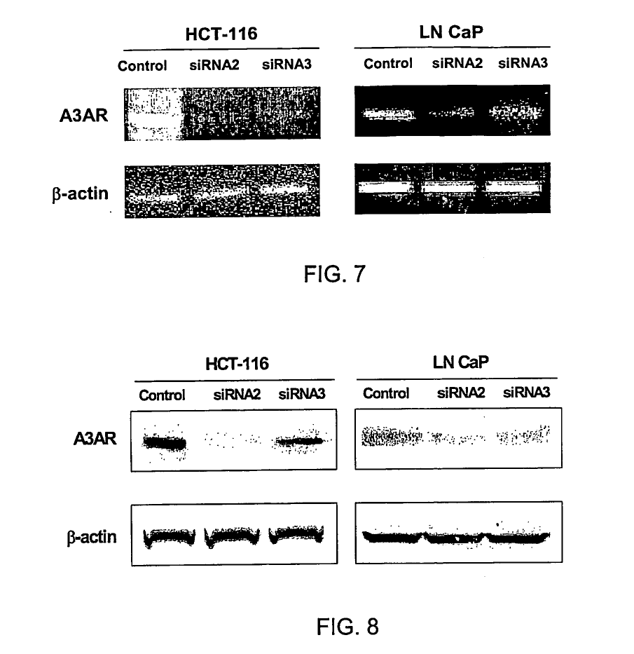

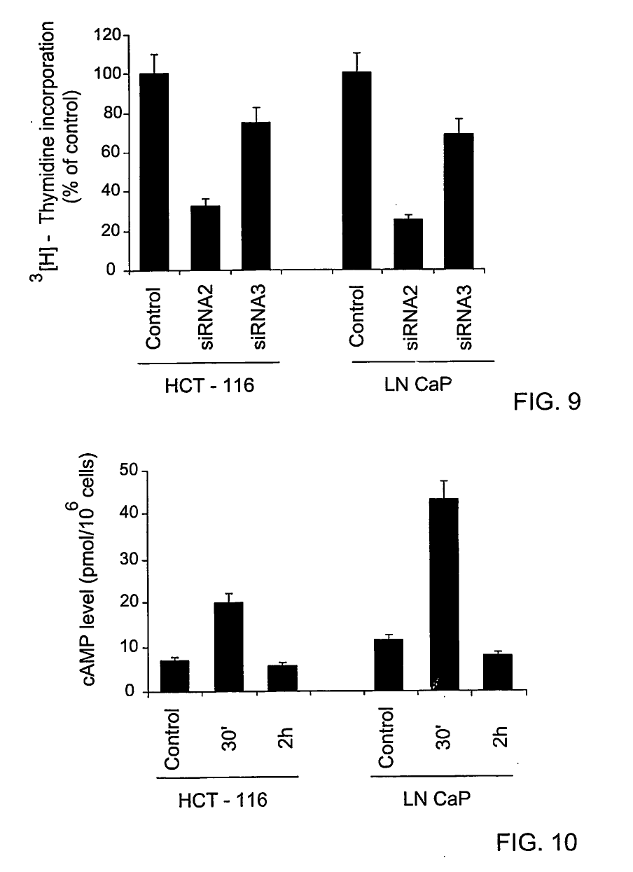

The above results were reproducible in the HCT-116 colon and LNCaP prostate carcinoma cells. These two human cell lines were transfected with siRNA2 and siRNA3 duplexes. RT-PCR analysis revealed that both siRNA duplexes decreased A₃AR mRNA and protein expression level in the 2 cell lines. The siRNA2 duplex was more effective in the silencing of A₃AR compared to siRNA3 duplex (FIG. 7 and 8). The degree of growth inhibition was directly correlated to the ability of each duplex, siRNA2 or siRNA3, to silence A₃AR. For the HCT-116 cells 75%±6% inhibition was observed utilizing siRNA2 and 32.9%±2% inhibition for the siRNA3. Similarly, in the LNCaP prostate cancer cells 75%±3% inhibition was seen with siRNA2 and 25%+2% with siRNA3 (FIG. 9).

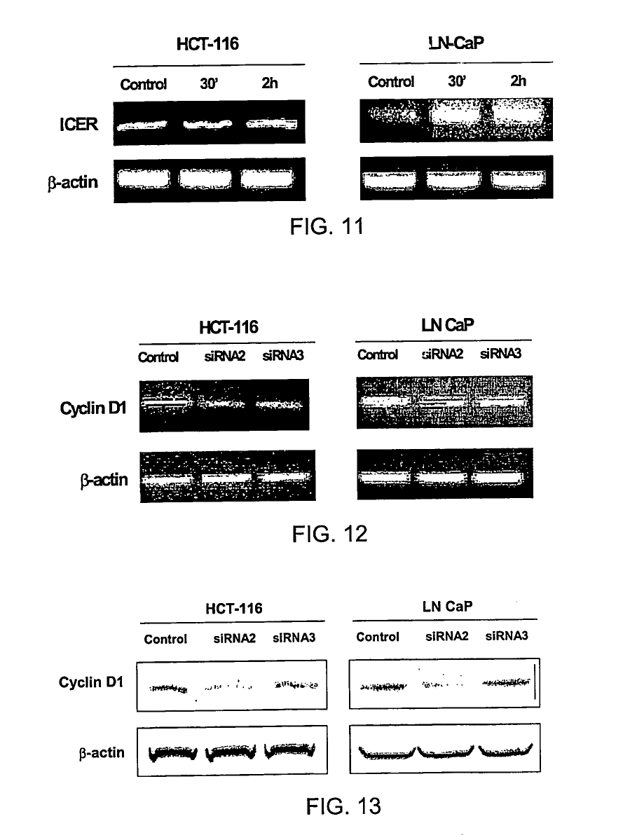

To examine the involvement of the cAMP signaling pathway in mediating the inhibitory effect of A₃AR siRNA transfection on tumor growth, cAMP level and down-stream proteins involved with the transmission of the signal were assessed. A similar response to that observed with the silenced B16-F10 melanoma cells was observed. In HCT-116 and LN-CaP cells transfected with A₃AR siRNA2, marked increase in cAMP formation (FIG. 10) was noted 30 min after transcription. An up-regulation of the expression level of ICER mRNA was also seen in the 2 cell lines (FIG. 11).

A decline in the mRNA (FIG. 12) and protein expression level (FIG. 13) of cyclin D1 was observed in the A₃AR silenced colon and prostate cells indicating that the modulation of the cAMP responsive transcription factors had a functional effect.

Stable Transfection

siRNA target sequences are selected similarly to the target selection process for a non-stable transfection as described above. The target sequence may, for example, include 19 nucleotides. However, as known, the length of the target sequence may be other than 19, e.g. in the range of 17-24. This target sequence may be flanked with AA at the 5′ and with TT at the 3′ ends. However, good results can also be obtained with the sequences that are flanked with either AA or TT at both the 5′ and 3′ ends. The target sequence to be flanked is preferably from the coding region of the A3AR MRNA, typically at least 100 bp from either the start or termination of the MRNA translation. The GC content of the target sequence should preferably be more than 30%.

The following is an example of a designed siRNA target sequence:

| | |
|---|---|
| AAGGGTGCCTAGTTGACTTAC | [SEQ ID No: 18] |

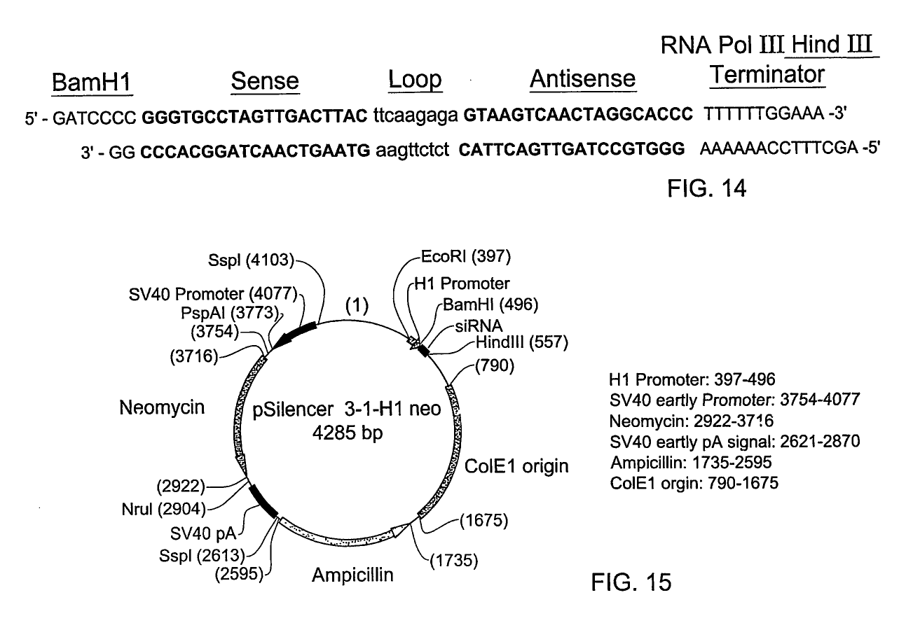

An example of a designed oligonucleotide duplex that includes flanking BamH1 and Hind III restriction sites, a sense and an antisense coding sequence, a loop sequence and RNA Pol III terminator sequence and that can be incorporated eventually in the plasmid is shown in FIG. 14.

Such designed oligonucleotides can then be cloned into a plasmid such as the pSilencer 3.1-H1 neo (Ambion) under H1 promoter, shown in FIG. 15. An example of a plasmid preparation and cloning protocol is shown below.

Protocol for Cloning:

Annealing of oligonucleotides (“oligos”)
dissolve oligos in 50 μl H₂O (gives about 3 μg/μl)
take 1 μl from each oligo (forward+reverse)
add 48 μl annealing buffer (100 mM potassium acetate; 30 mM HEPES-KOH pH 7.4; 2 mM Mg-acetate)
incubate 4 minutes at 95° C.
incubate 10 minutes at 70° C.
slowly cool down the annealed oligos to 4° C. (or 10° C.)
Cooled samples can be stored at −20° C.

Phosphorylation of Oligos
take 2 μl of the annealed oligos
add 1 μl T4 PNK (polynucleotide kinase) buffer
add 1 μl 10 mM ATP (final is 1 mM)
add 1 μl T4 PNK
add 5 μl H₂O
incubate 30 minutes at 37° C.
incubate 10 minutes at 70°C. (heat inactivation step PNK)

Ligation into pSilencer 3.1-H1 neo
take 2 μl of the annealed phosphorylated oligos
add 1 μl ligase buffer
add 1 μl pSilencer digested with BamH1 and HindIII
add 5 μl H₂O
add 1 μl ligase
incubate 1 hour at RT
   Transform Bacteria
   Grow Minipreps and Check Insert with BamH1-HindIII Digestion

1. A short interfering RNA (siRNA) duplex that includes complementary sense and anti-sense sequences corresponding to at least part of the A3 adenosine receptor (A3AR) mRNA sequence, or an alternative splice form, mutant or cognate thereof.

2. A double-stranded RNA (dsRNA) construct that can be converted within a cell into a siRNA duplex of claim 1.

3. A transcript system that can induce transcription within cells of either a siRNA duplex according to claim 1 or a dsRNA construct according to claim 2.

4. The siRNA duplex of claim 1 wherein the sense sequence is identical to at least part of the A3AR mRNA sequence and the antisense sequence is complementary to at least part of the A3AR mRNA sequence.

5. The siRNA duplex of claim 1 wherein the sense or antisense sequences sufficiently correspond to at least part of the A3AR mRNA sequence so as to activate RNA interference-based cleavage of the mRNA sequence.

6. The siRNA duplex of claim 1 which includes at least one of the following features:
(a) the sense and antisense sequences in the siRNA duplex that can pair with one another include approximately 20 to 22 nucleotides within the coding region of the A3AR mRNA;
(b) at least one of said sense or antisense sequences in the siRNA duplex have a short tail of poly T;
(c) at least one of the sense or antisense sequences in the siRNA duplex have no G-nucleotide at the 3′ end;
(d) the GC contents is less than 50%; and
(e) the sequences are unique to the A3 adenosine receptor and have no similarity to the other subtypes of adenosine receptors.

7. A method for treating a hyperproliferative disease comprising contacting hyperproliferating cells with an active agent being the siRNA duplex of claim 1.

8-19. (canceled)

20. A method for treating a hyperproliferative disease comprising contacting hyperproliferating cells with an active agent being the dsRNA construct of claim 2.

21. A method for treating a hyperproliferative disease comprising contacting hyperproliferating cells with an active agent being the transcript system of claim 3.

22. A pharmaceutical composition for the treatment of hyperproliferative disease comprising as active agent an effective amount of the siRNA duplex of claim 1.

23. A pharmaceutical composition for the treatment of hyperproliferative disease comprising as active agent an effective amount of the dsRNA construct of claim 2.

24. A pharmaceutical composition for the treatment of hyperproliferative disease comprising as active agent an effective amount of the transcript system of claim 3.

25. A method for inhibiting expression of the A3AR gene in target cells comprising introducing an active agent into said target cells, wherein said active agent is selected from the group consisting of:
(a) an siRNA duplex that includes complementary sense and anti-sense sequences corresponding to at least part of the A3 adenosine receptor (A3AR) mRNA sequence, or an alternative splice form, mutant or cognate thereof;
(b) a dsRNA construct that can be converted within a cell into a siRNA duplex of (a); and
(c) a transcript system that can induce transcription within cells of either a siRNA duplex according to. (a) or a dsRNA construct according to (b).

26. The method of claim 25 in which the A3AR gene expression is inhibited by at least 25%.

27. The method of claim 25 wherein the active agent is transfected into the target cells by a delivery system.

28. A method of activating A3AR-specific RNA interference in a target cell comprising introducing into said target cells an active agent, wherein said active agent is selected from the group consisting of:
(a) an siRNA duplex that includes complementary sense and anti-sense sequences corresponding to at least part of the A3 adenosine receptor (A3AR) mRNA sequence, or an alternative splice form, mutant or cognate thereof;
(b) a dsRNA construct that can be converted within a cell into a siRNa duplex of (a); and
(c) a transcript system that can induce transcription within cells of either a siRNA duplex according to (a) or a dsRNA construct according to (b).

29. A recombinant plasmid comprising nucleic acid sequences for expressing one or more of the sequences comprising the siRNA duplex of claim 1.

30. A kit comprising reagents for activating A3AR-specific RNA interference in a cell or organism.

31. The siRNA duplex of claim 1 comprising the following respective sense and antisense sequences:

| | |
|---|---|
| r(GUGACCCACCUGUGAUGAG)d(TT) | [SEQ ID No: 5] |
| and | |
| r(CUCAUCACAGGUGGGUCAC)d(TT). | [SEQ ID No: 6] |

32. The siRNA duplex of claim 1 comprising the following respective sense and antisense sequences:

| | |
|---|---|
| r(GGGUGCCUAGUUGACUUAC)d(TT) | [SEQ ID No: 8] |
| and | |
| r(GUAAGUCAACUAGGCACCC)d(TT). | [SEQ ID No: 9] |


SEQUENCE LISTING (18 sequences)

SEQ ID NO: 1 - DNA, 21 bp, Homo sapiens
aacggttaccactcaaagaag

SEQ ID NO: 2 - DNA, 23 bp, Homo sapiens
rcgguuaccacucaaagaagdtt

SEQ ID NO: 3 - DNA, 23 bp, Homo sapiens
rcuucuuugagugguaaccgdtt

SEQ ID NO: 4 - DNA, 21 bp, Homo sapiens
aagtgacccacctgtgatgag

SEQ ID NO: 5 - DNA, 23 bp, Homo sapiens
rgugacccaccugugaugagdtt

SEQ ID NO: 6 - DNA, 23 bp, Homo sapiens
rcucaucacaggugggucacdtt

SEQ ID NO: 7 - DNA, 21 bp, Homo sapiens
aagggtgcctagttgacttac

SEQ ID NO: 8 - DNA, 23 bp, Homo sapiens
rgggugccuaguugacuuacdtt

SEQ ID NO: 9 - DNA, 23 bp, Homo sapiens
rguaagucaacuaggcacccdtt

SEQ ID NO: 10 - DNA, 23 bp, Homo sapiens
acggtgaggtaccacagcttgtg

SEQ ID NO: 11 - DNA, 20 bp, Homo sapiens
ataccgcgggatggcagacc

SEQ ID NO: 12 - DNA, 19 bp, Homo sapiens
ccgagaaggggaagacagg

SEQ ID NO: 13 - DNA, 20 bp, Homo sapiens
tgctatattcttcccccaag

SEQ ID NO: 14 - DNA, 25 bp, Homo sapiens
gaacaaacagatcatccgcaaacac

SEQ ID NO: 15 - DNA, 24 bp, Homo sapiens
gctcctggcaggcccggaggcagt

SEQ ID NO: 16 - DNA, 20 bp, Homo sapiens
aacttcctctcctgctaccg

SEQ ID NO: 17 - DNA, 20 bp, Homo sapiens
gtggctcccgcctgcccggt

SEQ ID NO: 18 - DNA, 21 bp, Homo sapiens
aagggtgcctagttgacttac